(19) United States
(12) Patent Application Publication
(10) Pub. No.: US 2015/0114424 A1
(43) Pub. Date: Apr. 30, 2015

(54) SHAPING AGENT FOR KERATINOUS FIBRES AND HAIR SHAPING METHOD

(71) Applicant: Henkel AG & Co., KGaA, Dusseldorf (DE)

(72) Inventors: Wolfgang Wolff, Bargteheide (DE); Yvonne Lissner, Hamburg (DE); Birgit Rautenberg-Groth, Ellerau (DE); Hartmut Manneck, Barnitz (DE); Mustafa Akram, Hamburg (DE)

(73) Assignee: Henkel AG & Co. KGaA, Dusseldorf (DE)

(21) Appl. No.: 14/367,808

(22) PCT Filed: Nov. 21, 2012
(86) PCT No.: PCT/EP2012/073168

§ 371 Date: Jun. 20, 2014

(30) Foreign Application Priority Data
Dec. 22, 2011 (DE) 10 2011 089 573.6

(51) Int. Cl. A61K 8/81; A61K 8/23; A45D 7/06; A61Q 5/06

(52) U.S. Cl. CPC A61K 8/8111; A61Q 5/06; A61K 8/23; A45D 7/06
USPC 132/206; 424/70.11

(57) ABSTRACT

A method is provided for the lasting reshaping of keratin-containing fibers, in particular human hair, in which i) a shaping agent containing 1) keratin-reducing substance(s) and 2) a polyisoprene is applied to the fibers, ii) the shaping agent acts for a contact time (Z1), and the fibers are shaped under the action of heat.

### CROSS REFERENCED TO RELATED APPLICATIONS

The present disclosure is a U.S. National Stage entry under 35 U.S.C. §371 based on International Application No. PCT/EP2012/073168, filed Nov. 21, 2012 which was published under PCT Article 21(2) and which claims priority to German Patent Application No. DE 10 2011 089 573.6 filed on Dec. 22, 2011, which is incorporated herein by reference in its entirety.

### TECHNICAL FIELD

The technical field relates to a method for the lasting shaping of keratinic fibers, in particular for the straightening of keratinic fibers, and to shaping agents for performing this method.

### BACKGROUND

The cosmetic, lasting shaping of keratinic fibers is based essentially on the mechanical reshaping of the chemically pretreated fibers. The fibers that are reshaped in this way are then optionally fixed by means of a suitable fixing agent.

During this method the keratinic fibers are treated with an aqueous keratin-reducing substance. The keratin-reducing substance cleaves off some of the disulfide bonds of the keratin to —SH groups, resulting in a loosening of the peptide crosslinking and a reorientation of the keratin structure due to the tensioning of the fibers by the mechanical shaping. The aqueous preparation of the keratin-reducing substance is conventionally made alkaline in order to allow a deep penetration of the keratin-reducing substance into the fibers through swelling of the fibers and to ensure an adequate deprotonation of the thiol functions of the hair.

The mechanical reshaping, performed in parallel or afterwards, leads to a reorientation of the keratin structure in the hair. In a subsequent step the fibers can be treated with the aqueous preparation of an oxidizing agent. Under the influence of the oxidizing agent new disulfide bonds are formed, and in this way the keratin structure is newly fixed in the defined shape.

The above method can be used both to create curls and waves in straight hair and to straighten curly hair. However, the results obtained with this method, in particular with regard to hair straightening, leave room for improvement.

Hair treatment agents having a high concentration of formaldehyde or methylene glycol are offered as an alternative for intensive hair straightening. However, the use of such structure-changing agents based on formaldehyde and formaldehyde-releasing compounds is banned under the European Cosmetics Directive because of the associated health risks.

As an alternative to the formaldehyde-containing agents, the use of oxoacetamide carbocysteine has been discussed recently in the area of hair-straightening agents. However, the effectiveness of these agents varies considerably according to the hair type.

Accordingly, at least one object herein is to provide hair-shaping methods and hair-shaping agents which are suitable in particular for hair straightening while being equally effective on different hair types and posing no health risks. Furthermore, other desirable features and characteristics will become apparent from the subsequent detailed description of the invention and the appended claims, taken in conjunction with this background of the invention.

### SUMMARY

In accordance with exemplary embodiments, shaping agents for keratinic fibers and methods for the lasting reshaping of keratin-containing fibers, in particular human hair, are provided. In an exemplary embodiment, a method is provided for the lasting reshaping of keratin-containing fibers, in particular human hair, in which

i. a shaping agent comprising
1. keratin-reducing substance(s), and
2. a polyisoprene
is applied to the fibers,
   ii. the shaping agent acts for a contact time Z1, and
   iii. the fibers are shaped under the action of heat.

In another exemplary embodiment, a shaping agent is provided that includes:

1. keratin-reducing substance(s); and
2. a polyisoprene.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

The agents used in the method provided here contain the active agents preferably in a cosmetic carrier. Within the meaning herein, this cosmetic carrier is aqueous, alcoholic or aqueous-alcoholic. Within the meaning herein, aqueous-alcoholic carriers are understood to be hydrous compositions containing, relative to their total weight, about 0.5 to about 30 wt. %, preferably from about 2.5 to about 25 wt. % and in particular from about 5 to about 20 wt. % of a C₁-C₄ alcohol, preferably ethanol and/or 2-propanol and/or 1,2-propylene glycol. An aqueous carrier contains within the meaning herein, at least about 30 wt. %, in particular at least about 50 wt. %, of water, relative to the total weight of the application mixture. Preferred agents contain, relative to their total weight, about 40 to about 99 wt. %, preferably about 50 to about 98 wt. %, particularly preferably about 60 to about 95 wt. % and in particular about 70 to about 90 wt. % of water. The pH (10% solution, 20° C.) of preferred agents is about 2 to about 12, preferably about 7 to about 11 and in particular about 8 to about 10. Preferred methods herein include those in which the shaping agent has a pH (10% solution in water, 20° C.) from about 2 to about 12, preferably from about 7 to about 11 and in particular from about 8 to about 10.

With regard to the feasibility of the methods provided herein and with regard to the applicability of the agents provided herein, it has proved advantageous for the shaping agent to have under normal conditions (20° C., 1013.25 mbar) a viscosity (Brookfield RTV, spindle 4, 20 rpm) from about 7.5 to about 500 Pas, preferably from about 10 to about 400 Pas, particularly preferably from about 12.5 to about 300 Pas, more preferably from about 15 to about 200 Pas, still more preferably from about 17.5 to about 100 Pas, most particularly preferably from about 20 to about 100 Pas, more preferably from about 21 to about 75 Pas and in particular from about 22.5 to about 50 Pas.

The agents provided herein and the agents used in the methods provided herein contain as a first constituent keratin-reducing substance(s). Sulfites, preferably alkali, ammonium and/or alkanol ammonium salts of sulfurous acid and disulfurous acid, in particular sodium sulfite (Na₂SO₃) and/or sodium pyrosulfite (Na₂S₂O₅), mercaptans, preferably Bunte salts, thioglycolic acid, thiolactic acid, thiomalic acid, mercaptoethanesulfonic acid and salts and esters thereof, cysteamine, cysteine, preferably the alkali or ammonium salts of thioglycolic acid and/or thiolactic acid, are preferably used as keratin-reducing substance(s).

Preferred shaping agents contain the keratin-reducing substance(s) in amounts from about 1 to about 30 wt. %, preferably about 2.5 to about 25 wt. %, particularly preferably about 3 to about 20 wt. % and in particular about 5 to about 15 wt. %, relative in each case to their total weight.

As a second constituent the agents provided herein and the agents used in the methods provided herein contain polyisoprene. The use of the polyisoprene improves the results achieved in hair shaping, in particular in hair straightening, with regard both to the degree of shaping and to the holding duration of the shaping.

With regard to the cosmetic result, in particular to the degree of shaping and the holding duration of the shaping, the use of polyisoprene having a molecular weight from about 5×10² to about 5×10⁶ gmol⁻¹, preferably from about 10³ to about 4×10⁶ gmol⁻¹, particularly preferably from about 10⁴ to about 3×10⁶ gmol⁻¹ and in particular from about 10⁵ to about 3×10⁶ gmol⁻¹ has proved advantageous. The proportion by weight of polyisoprene in the total weight of the shaping agent is preferably about 0.01 to about 10 wt. %, preferably about 0.1 to about 8.0 wt. % and in particular about 0.5 to about 6.0 wt. %.

Preferred methods provided herein include those in which the contact time Z1 is about 1 to about 60 minutes, preferably about 2 to about 50 minutes, particularly preferably about 5 to about 40 minutes and in particular about 10 to about 30 minutes.

In the methods provided herein the keratin-containing fibers are shaped during or on completion of the contact time under the action of heat. It is preferable that in step iii. the keratinic fibers undergo a heat treatment using a heat source, wherein the heat source has a temperature from about 50 to about 200° C., preferably from about 90 to about 180° C. and in particular from about 120 to about 160° C. This is the case in particular if curling irons or straightening irons are used. Uses or methods provided herein, wherein the shaping takes place under the action of heat from a heat source on the keratinic fibers, the heat source having a temperature from about 50 to about 200° C., preferably from about 90 to about 180° C. and in particular from about 120 to about 160° C., are preferred.

If curling irons are used, a strand of hair is wound around a correspondingly heated rounded body, for example a rod-shaped or tubular body, and after a holding time—in particular of about 10 to about 30 seconds—is unwound again.

Particularly preferred uses or methods include those in which the shaping of the hair involves a straightening, preferably using straightening irons. As used herein, a mechanical straightening is understood to be a stretching of curly hair along the longest spatial extension of the hair fiber.

The hair-shaping agent can contain further ingredients. These are described below.

To improve the cosmetic effect and applicability, it is advantageous to incorporate polymers into the hair-shaping agents. The polymers that can be used to particular advantage in the methods and agents provided herein are described below:

Particularly preferred hair-shaping agents provided herein contain a copolymer B, containing
a structural unit according to the formula (B-I), in which
   R denotes a C₁ to C₃₀ alkyl group, a C₁ to C₄ aralkyl group, a C₂ to C₆ alkenyl group or a C₂ to C₆ hydroxyalkyl group, and
   X⁻ denotes a physiologically acceptable anion,
and a further structural unit according to formula (B-II), in which n denotes 1, 2 or 3 as the number of methylene units.

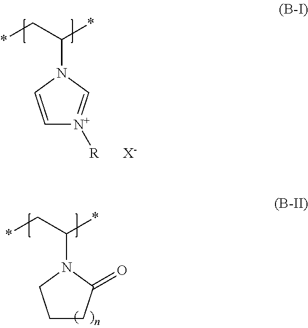

Film-forming and/or fixing copolymers B are known. These copolymers have a structural unit according to formula (B-I) and a structural unit according to formula (B-II) and can moreover have further structural units incorporated by the addition of corresponding monomers during polymerization.

In formula (B-I) R denotes a C₁ to C₃₀ alkyl group, a C₁ to C₄ aralkyl group, a C₂ to C₆ alkenyl group or a C₂ to C₆ hydroxyalkyl group. Preferred R groups are for example —CH₃; —CH₂CH₃, —CH₂CH₂CH₃, CH(CH₃)₂, —(CH₂)₃CH₃, —CH₂—CH(CH₃)₂, CH(CH₃)CH₂CH₃, —C(CH₃)₃, —CH₂OH, —CH₂CH₂OH, —CH₂CH₂CH₂OH, —CH(OH)CH₂CH₃, —CH₂CH(OH)CH₃.

X⁻ denotes a physiologically acceptable anion; preferred anions are chloride, bromide, iodide, sulfate, methosulfate, ethyl sulfate, tosylate and tetrafluoroborate.

In formula (B-II) n denotes the number of methylene groups. If n=1, formula (B-II) denotes a vinylpyrrolidone unit, if n=2 it denotes a vinylpiperidinone unit and if n=3 it denotes a vinylcaprolactam unit.

Particularly preferred hair-shaping agents provided herein include those in which as copolymer B they contain a copolymer B1, containing
a structural unit according to formula (B-I), in which R denotes a methyl group and X denotes methosulfate,
a further structural unit according to formula (B-II), in which n denotes 1 methylene unit.

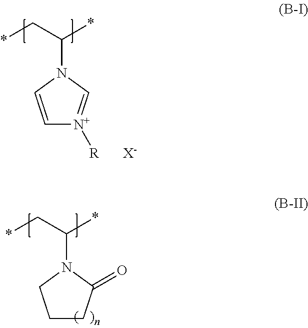

Most particularly preferred copolymers B1 contain about 10 to about 30 mol %, preferably about 15 to about 25 mol % and in particular about 20 mol % of structural units according to formula (B-I) and about 70 to about 90 mol %, preferably about 75 to about 85 mol % and in particular about 80 mol % of structural units according to formula (B-II).

It is particularly preferable here for the copolymers B1 to contain, in addition to polymer units resulting from the incorporation of the cited structural units according to formula (B-I) and (B-II) in the copolymer, a maximum of about 5 wt. %, preferably a maximum of about 1 wt. %, of polymer units resulting from the incorporation of other monomers. The copolymers B1 are preferably made up exclusively of structural units of formula (B-I) and (B-II) and can be described by the general formula

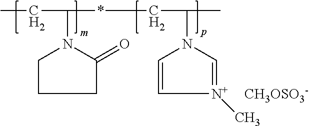

in which the indices m and n vary according to the molar mass of the polymer and are not intended to suggest that these are block copolymers. Structural units of formula (B-I) and formula (B-II) can rather be randomly distributed in the molecule.

Such N-methyl vinylimidazole/vinylpyrrolidone copolymers are referred to under INCI nomenclature as POLYQUATERNIUM-44 and are available for example from BASF under the trade name Luviquat® UltraCare.

Particularly preferred hair-shaping agents provided herein contain a copolymer B1 having molar masses within a defined range. Hair-shaping agents provided herein are preferred in which the copolymer B1 has a molar mass from about 50 to about 400 kDa, preferably from about 100 to about 300 kDa, more preferably from about 150 to about 250 kDa and in particular from about 190 to about 210 kDa.

In addition to the copolymer(s) B1 or in its or their place, the hair-shaping agents provided herein can also contain copolymers B2, which as additional structural units contain structural units of the formula (B-II), in which n denotes the number 3.

Further particularly preferred hair-shaping agents include those in which as copolymer B they contain a copolymer B2, containing
a structural unit according to formula (B-I), in which R denotes a methyl group and X denotes methosulfate,
a further structural unit according to formula (B-II), in which n denotes 1 methylene unit,
a further structural unit according to formula (B-II), in which n denotes 3 methylene units.

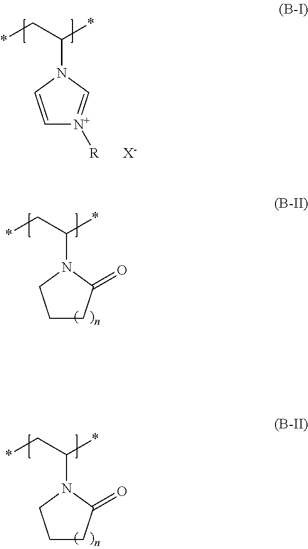

Here too it is particularly preferable for the copolymers B2 to contain, in addition to polymer units resulting from the incorporation of the cited structural units according to formula (B-I) and (B-II) in the copolymer, a maximum of about 5 wt. %, preferably a maximum of about 1 wt. %, of polymer units resulting from the incorporation of other monomers. The copolymers B2 are preferably made up exclusively of structural units of formula (B-I) and (B-II) and can be described by the general formula

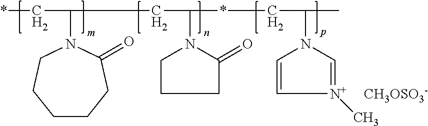

in which the indices m, n and p vary according to the molar mass of the polymer and are not intended to suggest that these are block copolymers. Structural units of formula (B-I) and formula (B-II) can rather be randomly distributed in the molecule.

Such N-methyl vinylimidazole/vinylpyrrolidone/vinylcaprolactam copolymers are referred to under NCI nomenclature as POLYQUATERNIUM-46 and are available for example from BASF under the trade name Luviquat® Hold.

Most particularly preferred copolymers B2 contain about 1 to about 20 mol.%, preferably about 5 to about 15 mol.% and in particular about 10 mol % of structural units according to formula (B-I) and about 30 to about 50 mol %, preferably about 35 to about 45 mol % and in particular about 40 mol % of structural units according to formula (B-II) with n=1 and about 40 to about 60 mol %, preferably about 45 to about 55 mol % and in particular about 60 mol % of structural units according to formula (B-II) with n=3.

Particularly preferred hair-shaping agents contain a copolymer B2 having molar masses within a defined range. Hair-shaping agents are preferred here in which the copolymer B2 has a molar mass from about 100 to about 1000 kDa, preferably from about 250 to about 900 kDa, more preferably from about 500 to about 850 kDa and in particular from about 650 to about 710 kDa.

In addition to the copolymer(s) B1 and/or B2 or in its or their place, the hair-shaping agents can also contain copolymers B3, which as additional structural units contain structural units of the formula (B-II), in which n denotes the number 3, and further distructural units from the group of vinylimidazole units and further structural units from the group of acrylamide and/or methacrylamide units.

Further particularly preferred hair-shaping agents include those in which as copolymer B they contain a copolymer B3, containing
a structural unit according to formula (B-I), in which R denotes a methyl group and X denotes methosulfate,
a further structural unit according to formula (B-II), in which n denotes 1 methylene unit,
a further structural unit according to formula (B-III),
a further structural unit according to formula (B-IV)

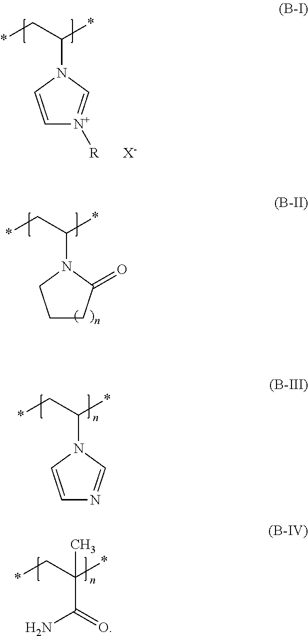

Here too it is particularly preferable for the copolymers B3 to contain, in addition to polymer units resulting from the incorporation of the cited structural units according to formula (B-I), (B-II), (B-III) and (B-IV) in the copolymer, a maximum of about 5 wt. %, preferably a maximum of 1 wt. %, of polymer units resulting from the incorporation of other monomers. The copolymers B3 are preferably made up exclusively of structural units of formula (B-I), (B-II), (B-III) and (B-IV) and can be described by the general formula

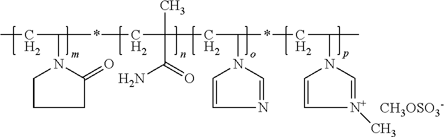

in which the indices m, n, o and p vary according to the molar mass of the polymer and are not intended to suggest that these are block copolymers. Structural units of formulae (B-I), (B-II), (B-III) and (B-IV) can rather be randomly distributed in the molecule.

Such N-methyl vinylimidazole/vinylpyrrolidone/vinylimidazole/methacrylamide copolymers are referred to under NCI nomenclature as POLYQUATERNIUM-68 and are available for example from BASF under the trade name Luviquat® Supreme.

Most particularly preferred copolymers B3 contain about 1 to about 12 mol.%, preferably about 3 to about 9 mol.% and in particular about 6 mol % of structural units according to formula (B-I) and about 45 to about 65 mol %, preferably about 50 to about 60 mol % and in particular about 55 mol % of structural units according to formula (B-II) with n=1 and about 1 to about 20 mol %, preferably about 5 to about 15 mol % and in particular about 10 mol % of structural units according to formula (B-III) and about 20 to about 40 mol %, preferably about 25 to about 35 mol % and in particular about 29 mol % of structural units according to formula (B-IV).

Particularly preferred hair-shaping agents contain a copolymer B3 having molar masses within a defined range. Hair-shaping agents are preferred here in which the copolymer B3 has a molar mass from about 100 to about 500 kDa, preferably from about 150 to about 400 kDa, more preferably from about 250 to about 350 kDa and in particular from about 290 to about 310 kDa.

Irrespective of whether just one copolymer B or several copolymers B are used and irrespective of the choice of the specific copolymer B, hair-shaping agents are preferred in which the total amount of copolymers B, relative to the weight of the ready-to-use hair-shaping agent, is about 0.05 to about 5 wt. %, preferably about 0.1 to about 4 wt. % and in particular about 0.25 to about 3 wt. %.

As an alternative to or in addition to the aforementioned polymers, preferred hair-shaping agents contain a copolymer F, selected from
f1) copolymers of vinylpyrrolidone with methacrylamidopropyl trimethylammonium chloride (MAPTAC) and/or
f2) copolymers of vinylpyrrolidone with dimethylaminoethyl methacrylate and/or
f3) copolymers of vinylpyrrolidone with dimethylaminopropyl methacrylamide and alkyl dimethylpropyl methacrylamidoammonium salts.

Thus hair-shaping agents are preferred for example which contain as polymer F copolymers of vinylpyrrolidone with methacrylamidopropyl trimethylammonium chloride (MAPTAC) (bl).

These can be described by the general formula

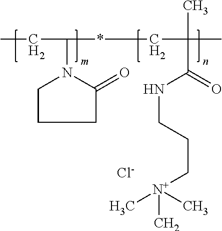

in which the indices m and n vary according to the molar mass of the polymer and are not intended to suggest that these are block copolymers. The structural units can rather be randomly distributed in the molecule.

Particularly preferred hair-shaping agents include those in which as the cationic polymer f1 they contain copolymers of methacrylamidopropyl trimethylammonium chloride (MAPTAC) with vinylpyrrolidone, containing about 40 to about 95 mol %, preferably about 42.5 to about 90 mol %, more preferably about 45 to about 85 mol % and in particular about 50 to about 80 mol % of vinylpyrrolidone.

Particularly preferred hair-shaping agents are further include those in which the copolymers f1 have molar masses from about 10 to about 1000 kDa, preferably from about 25 to about 900 kDa, more preferably from about 50 to about 800 kDa and in particular from about 100 to about 750 kDa.

The copolymers f1 are preferably used within defined quantity ranges. Hair-shaping agents are preferred here which, relative to the weight of the ready-to-use hair-shaping agent, contain about 0.05 to about 5 wt. %, preferably about 0.1 to about 4 wt. % and in particular about 0.25 to about 3 wt. % of copolymer(s) f1.

A most particularly preferred copolymer f1 is referred to under NCI nomenclature as Polyquaternium-28. Such a polymer is available for example under the trade name Gafquat® HS-100 (ISP).

In addition to the polymer(s) f1 or in its or their place, the hair-shaping agents can also contain polymers f2 from the group of copolymers of vinylpyrrolidone with dimethylaminoethyl methacrylate.

These can be described by the general formula

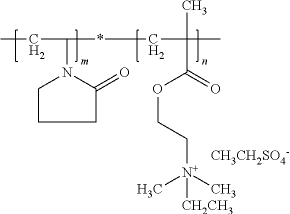

in which the indices m and n vary according to the molar mass of the polymer and are not intended to suggest that these are block copolymers. The structural units can rather be randomly distributed in the molecule.

Particularly preferred hair-shaping agents include those in which as the cationic polymer f2 they contain copolymers of vinylpyrrolidone with dimethylaminoethyl methacrylate, containing about 40 to about 95 mol %, preferably about 42.5 to about 90 mol %, more preferably about 45 to about 85 mol % and in particular about 50 to about 80 mol % of vinylpyrrolidone.

Particularly preferred hair-shaping agents include those in which the copolymers f2 have molar masses from about 100 to about 2500 kDa, preferably from about 250 to about 2000 kDa, more preferably from about 500 to about 1750 kDa and in particular from about 800 to about 1500 kDa.

The copolymers f2 are preferably used within defined quantity ranges. Hair-shaping agents are preferred here which, relative to the weight of the ready-to-use hair-shaping agent contain about 0.05 to about 5 wt. %, preferably about 0.1 to about 4 wt. % and in particular about 0.25 to about 3 wt. % of copolymer(s) f2.

A most particularly preferred copolymer f2 is referred to under NCI nomenclature as Polyquaternium-11. Such a polymer is available for example under the trade name Gafquat® 755 N (ISP).

In addition to the polymer(s) f1 and/or the polymer(s) f2 or in its or their place, the hair-shaping agents provided herein can also contain polymers f3 from the group of copolymers of vinylpyrrolidone with dimethylaminopropyl methacrylamide and alkyl dimethylpropyl methacrylamidoammonium salts.

These can be described by the general formula

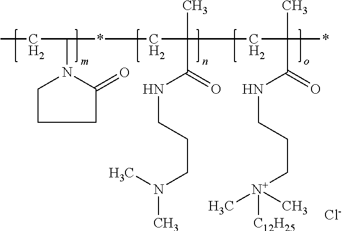

in which the indices m, n and o vary according to the molar mass of the polymer and are not intended to suggest that the copolymers are block copolymers. The structural units can rather be randomly distributed in the molecule.

Particularly preferred hair-shaping agents include those in which as the cationic polymer f3 they contain copolymers of vinylpyrrolidone with dimethylaminopropyl methacrylamide and lauryl dimethylpropyl methacrylamidoammonium salts.

Particularly preferred hair-shaping agents include those in which as the cationic polymer f3 they contain copolymers of vinylpyrrolidone with dimethylaminopropyl methacrylamide and alkyl dimethylpropyl methacrylamidoammonium salts, containing about 40 to about 95 mol %, preferably about 42.5 to about 90 mol %, more preferably about 45 to about 85 mol % and in particular about 50 to about 80 mol % of vinylpyrrolidone.

Most particularly preferred hair-shaping agents include those in which the copolymers f3 have molar masses from about 10 to about 1000 kDa, preferably from about 25 to about 900 kDa, more preferably from about 50 to about 800 kDa and in particular from about 100 to about 750 kDa.

The copolymers f3 are preferably used within defined quantity ranges. Hair-shaping agents are preferred here which, relative to the weight of the ready-to-use hair-shaping agent, contain about 0.05 to about 5 wt. %, preferably about 0.1 to about 4 wt. % and in particular about 0.25 to about 3 wt. % of copolymer(s) f3.

A most particularly preferred copolymer f3 is referred to under NCI nomenclature as Polyquaternium-55. Such a polymer is available for example under the trade name Styleze® W20 (ISP).

The agents used in the method provided herein and the agents provided herein can contain further substances, in particular care substances.

Thus it is possible for example to incorporate cationic surfactants into the hair-shaping agents. Preferred hair-shaping agents additionally contain cationic surfactant(s), preferably in amounts from about 0.5 to about 15 wt. %, particularly preferably from about 1 to about 10 wt. %, and in particular from about 1.5 to about 7.5 wt. %, relative in each case to the total agent.

Cationic surfactants of the quaternary ammonium compound, esterquat and amidoamine type can be used in the agents provided herein. Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the NCI names Quaternium-27 and Quaternium-83. The long alkyl chains of the aforementioned surfactants preferably have 10 to 18 carbon atoms.

Quaternary ammonium compounds having behenyl residues, in particular the substances known under the name behentrimonium chloride or behentrimonium bromide (docosanyltrimethylammonium chloride or bromide), can preferably be used in the agents provided herein. Other preferred quaternary ammonium compounds have two behenyl residues, those having two behenyl residues on an imidazolinium backbone being particularly preferred. These substances are commercially available for example under the names Genamin® KDMP (Clariant) and Crodazosoft® DBQ (Crodauza).

Esterquats are known substances containing both an ester function and a quaternary ammonium group as a structural element. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines Such products are sold under the trademarks Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU-35 are examples of such esterquats.

The alkylamidoamines are conventionally produced by amidation of natural or synthetic fatty acids and fatty acid cuts with dialkyl amino amines. A compound from this group of substances that is particularly suitable for the agents provided herein is the stearamidopropyl dimethylamine which is commercially available under the name Tegoamid® S 18.

Further care substances which can advantageously be included in the hair-shaping agents are fats or cosmetic oil bodies. The amount of natural and synthetic cosmetic oil bodies used in the hair-shaping agents is conventionally about 0.1 to about 20 wt., preferably about 0.2 to about 10 wt. % and in particular about 0.25 to about 7.5 wt. %, relative to the total agent.

The preferred oil bodies include in particular:
vegetable oils. Examples of such oils are amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, maize oil, almond oil, manila oil, evening primrose oil, olive oil, orange oil, palm oil, peach kernel oil, rapeseed oil, rice oil, sea buckthorn fruit oil, sea buckthorn seed oil, sesame oil, shea butter, soybean oil, sunflower oil, grape seed oil, walnut oil, wheatgerm oil, wild rose oil and the liquid components of coconut oil. Other triglyceride oils are also suitable, however, such as the liquid components of beef fat and synthetic triglyceride oils. Preferred cosmetic agents provided herein include those in which the oil body b) is a natural oil. The proportion by weight of the natural oil in the total weight of preferred cosmetic agents is about 0.1 to about 10 wt. %, preferably about 0.2 to about 8.0 wt. % and in particular about 0.4 to about 5.0 wt. %.
liquid paraffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl)cyclohexane (Cetiol® S) and di-n-octyl ether (Cetiol® OE), can be preferred.
ester oils. Ester oils are understood to be the esters of C₆-C₃₀ fatty acids with C₂-C₃₀ fatty alcohols. The monoesters of fatty acids with alcohols having 2 to 24 C atoms are preferred. Particularly preferred herein are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V). Preferred cosmetic agents herein include those in which the oil body b) is an ester oil, preferably isopropyl myristate. The proportion by weight of the ester oil in the total weight of preferred cosmetic agents is about 0.1 to about 30 wt. %, preferably about 0.2 to about 27 wt. % and in particular about 0.4 to about 24 wt. %.
dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
fatty alcohols. The group of preferred fatty alcohols includes the C₈ to C₂₂ fatty alcohols, preferably the C₁₀ to C₂₀ fatty alcohols and in particular the C₁₂ to C₁₈ fatty alcohols. Preferred cosmetic agents herein include those in which the oil body b) is a fatty alcohol. The proportion by weight of the fatty alcohol in the total weight of preferred cosmetic agents is about 0.1 to about 10 wt. %, preferably about 0.2 to about 8.0 wt. % and in particular about 0.4 to about 5.0 wt. %.
symmetrical, asymmetrical or cyclic esters of carbonic acid with fatty alcohols, as described for example in DE-OS 197 56 454, glycerol carbonate or dicaprylyl carbonate (Cetiol® CC),
tri-fatty acid esters of saturated and/or unsaturated linear and/or branched fatty acids with glycerol,
fatty acid partial glycerides, namely monoglycerides, diglycerides and technical mixtures thereof. If technical products are used, small amounts of triglycerides may also be included for production reasons. The partial glycerides preferably obey formula (D4-I),

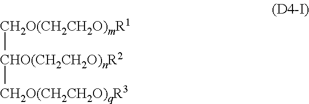

in which R¹, R² and R³ independently of one another denote hydrogen or a linear or branched, saturated and/or unsaturated acyl residue having 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. R¹ preferably denotes an acyl residue and R² and R³ hydrogen and the sum (m+n+q) is preferably 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

It has furthermore proved advantageous for polymers to be included in the hair-shaping agents as care substances, wherein cationic, anionic, amphoteric and non-ionic polymers have all proved effective. Preferred hair-shaping agents include those that contain additional polymer(s), preferably non-ionic and/or cationic polymer(s), preferably amounts from about 0.1 to about 20 wt. %, particularly preferably from about 0.2 to about 15 wt. % and in particular from about 0.3 to about 10 wt. %, relative in each case to the total agent.

Preferred cationic polymers are for example
quaternized cellulose derivatives such as are available commercially under the names Celquat® and Polymer JR®. The compounds Celquat® H 100, Celquat® L 200 and Polymer JR®400 are preferred quaternized cellulose derivatives,
cationic alkyl polyglycosides according to DE-PS 44 13 686,
cationized honey, for example the commercial product Honeyquat® 50,
cationic guar derivatives, such as in particular the products sold under the trade names Cosmedia®Guar and Jaguar®,
polymeric dimethyldiallyl ammonium salts and copolymers thereof with esters and amides of acrylic acid and methacrylic acid. The products available commercially under the names Merquat® 100 (poly(dimethyldiallyl ammonium chloride)) and Merquat® 550 (dimethyldiallyl ammonium chloride-acrylamide copolymer) are examples of such cationic polymers,
copolymers of vinylpyrrolidone with quaternized derivatives of dialkyl aminoalkyl acrylate and methacrylate, such as for example diethyl sulfate-quaternized vinylpyrrolidone-dimethyl aminoethyl methacrylate copolymers. Such compounds are available commercially under the names Gafquat®734 and Gafquat®755,
vinylpyrrolidone-vinylimidazolium methochloride copolymers, such as are sold under the names Luviquat® FC 370, FC 550, FC 905 and HM 552,
quaternized polyvinyl alcohol,
and the polymers known under the names Polyquaternium-2, Polyquaternium-17, Polyquaternium-18, Polyquaternium-24 and Polyquaternium-27 with quaternary nitrogen atoms in the polymer main chain.

Cationic polymers that are particularly preferred herein are cationic cellulose derivatives and chitosan and derivatives thereof, in particular the commercial products Polymer® JR 400, Hydagen® HCMF and Kytamer® PC, cationic guar derivatives, cationic honey derivatives, in particular the commercial product Honeyquat® 50, cationic alkyl polyglycosides according to DE-PS 44 13 686 and polymers of the Polyquaternium-37 type.

Protein hydrolysates of plant, animal, marine or synthetic origin can be used in the agents provided herein as polymeric cationic care substances.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Further protein hydrolysates that are preferred for the agents provided herein are of marine origin. They include for example collagen hydrolysates of fish or algae and protein hydrolysates of mussels or pearl hydrolysates. Examples of pearl extracts suitable for the agents provided herein are the commercial products Pearl Protein Extract BG® or Crodarom® Pearl.

Cationized protein hydrolysates are likewise included among the protein hydrolysates and derivatives thereof, wherein the underlying protein hydrolysate can derive from animal sources, for example from collagen, milk or keratin, from plant sources, for example from wheat, corn, rice, potatoes, soy or almonds, from marine life forms, for example from fish collagen or algae, or from protein hydrolysates obtained by biotechnology. The protein hydrolysates (P) are contained in the compositions in concentrations from about 0.001 wt. % to about 20 wt. %, preferably from about 0.05 wt. % to about 15 wt. % and most particularly preferably in amounts from about 0.05 wt. % to about 5 wt. %.

Suitable non-ionic polymers are for example:
vinylpyrrolidone/vinyl ester copolymers, such as are sold for example under the trademark Luviskol® (BASF). Luviskol® VA 64 and Luviskol® VA 73, both of which are vinylpyrrolidone/vinyl acetate copolymers, are likewise preferred non-ionic polymers;
cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules);
starch and derivatives thereof, in particular starch ethers, for example Structure® XL (National Starch), a multifunctional, salt-tolerant starch;
shellac;
polyvinylpyrrolidones, such as are sold for example under the name Luviskol® (BASF);
siloxanes. These siloxanes can be both water-soluble and water-insoluble. Both volatile and non-volatile siloxanes are suitable, wherein compounds whose boiling point under normal pressure is above about 200° C. are understood to be non-volatile siloxanes. Preferred siloxanes are polydialkyl siloxanes, such as for example polydimethyl siloxane, polyalkylaryl siloxanes, such as for example polyphenylmethyl siloxane, ethoxylated polydialkyl siloxanes as well as polydialkyl siloxanes containing amine and/or hydroxyl groups;
glycoside-substituted silicones.

It is also possible for the agents provided herein to contain a plurality of, in particular two, different polymers of the same charge and/or an ionic and an amphoteric and/or non-ionic polymer.

Depending on the further effects, in addition to the shaping performance and optionally the care performance, that are to be achieved with the agents used in the method according to the invention, they can contain further ingredients. These are described below.

The use of surfactants has proved to be particularly advantageous. Thus in a further preferred embodiment the agents according to the invention and the agents used in the methods according to the invention contain surfactants.

Alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and protein fatty acid condensates are suitable in particular as anionic surfactants.

Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytic surfactants are N-cocoalkyl aminopropionate, cocoacylaminoethyl aminopropionate and C₁₂-C₁₈ acyl sarcosine.

The alkylene oxide addition products with saturated linear fatty alcohols and fatty acids each containing about 2 to about 30 mol of ethylene oxide per mol of fatty alcohol or fatty acid have proved to be preferred non-ionic surfactants. Agents having outstanding properties are likewise obtained if they contain fatty acid esters of ethoxylated glycerol as non-ionic surfactants.

These compounds generally have the following parameters. The alkyl residue R contains 6 to 22 carbon atoms and can be both linear and branched. Primary linear aliphatic residues and aliphatic residues that are methyl-branched in the 2-position are preferred. Such alkyl residues are for example 1-octyl, 1-decyl, 1-lauryl, 1-myristyl, 1-cetyl and 1-stearyl. 1-Octyl, 1-decyl, 1-lauryl and 1-myristyl are particularly preferred. If oxo alcohols are used as starting materials, compounds having an odd number of carbon atoms in the alkyl chain dominate.

Most particularly preferred non-ionic surfactants are moreover the sugar surfactants. These can be included in the agents provided herein preferably in amounts from about 0.1 to about 20 wt. %, relative to the total agent. Amounts from about 0.5 to about 15 wt. % are preferred, with amounts from about 0.5 to about 7.5 wt. % being most particularly preferred.

The cationic surfactants for use in the agents provided herein have already been described above as care substances.

In a further preferred embodiment the agents used in the method provided herein can contain emulsifiers. The agents used in the method provided herein contain the emulsifiers preferably in amounts from about 0.1 to about 25 wt. %, in particular about 0.5 to about 15 wt. %, relative to the total agent. The agents used in the method provided herein can preferably contain a non-ionogenic emulsifier having an HLB value from about 8 to about 18. Non-ionogenic emulsifiers having an HLB value of about 10 to about 15 can be particularly preferred for the agents provided herein.

Finally the agents used in the method provided herein can also contain plant extracts. The extracts from green tea, almond, aloe vera, coconut, mango, apricot, lemon, wheat, kiwi and melon are most particularly preferred.

It can additionally prove advantageous for penetration auxiliaries and/or swelling agents to be included in the agents used in the method provided herein. They include for example urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

At the end of the contact time for the hair-shaping agent, the hair is optionally treated with a fixing agent after optionally being rinsed. Oxidizing agents, for example sodium bromate, potassium bromate, hydrogen peroxide, and the conventional stabilizers for stabilizing aqueous hydrogen peroxide preparations are a required constituent of such fixing agents. The pH of such aqueous H₂O₂ preparations, which conventionally contain approximately about 0.5 to about 15 wt. % and in the ready-to-use form generally approximately about 0.5 to about 3 wt. % of H₂O₂, is preferably around about 2 to about 6, in particular about 2 to about 4; it is adjusted with inorganic acids, preferably phosphoric acid. Bromate-based fixing agents conventionally contain the bromates in concentrations from about 1 to about 10 wt. % and the pH of the solutions is adjusted to about 4 to about 7. Fixing agents on an enzymatic basis (e.g. peroxidases) which contain little or no oxidizing agent, in particular H₂O₂, are likewise suitable. The fixing agents preferably contain at least about 50 wt. % of water.

In another embodiment, a hair-shaping kit is provided that contains

a) a hair-shaping agent as provided herein and described above; and
b) a fixing agent.

In another embodiment, a method is provided for the lasting reshaping of keratin-containing fibers, in particular human hair, in which

i. a shaping agent comprising:
1. keratin-reducing substance(s); and
2. a polyisoprene
is applied to the fibers,
   ii. the shaping agent acts for a contact time Z1,
   iii. the fibers are shaped under the action of heat;
   iv. then a fixing agent containing an oxidizing agent is applied to the fibers and rinsed off again after a contact time Z2.

Methods that are preferred herein where the fixing agent contains at least one of the aforementioned polymers, preferably in amounts such that the total polymer content of the fixing agents is about 1 to about 15 wt. %, preferably about 2.5 to about 12.5 wt. %, more preferably about 4 to about 10 wt. % and in particular about 5 to about 8 wt. %.

The contact time for the fixing agent also preferably lies within relatively narrow ranges. Preferred methods herein include those in which the contact time Z2 is about 1 to about 60 minutes, preferably about 2 to about 50 minutes, particularly preferably about 5 to about 40 minutes and in particular about 10 to about 30 minutes.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A method for the lasting reshaping of keratin-containing fibers, in particular human hair, in which:
i. a shaping agent comprising
1. keratin-reducing substance(s); and
2. a polyisoprene
is applied to the fibers,
ii. the shaping agent acts for a contact time Z1, and
iii. the fibers are shaped under the action of heat.

2. The method according to claim 1, wherein the keratin-reducing substance(s) is/are chosen from sulfites, mercaptans, cysteamine, cysteine, and/or the alkali or ammonium salts of thioglycolic acid and/or thiolactic acid.

3. The method according to claim 1, wherein the polyisoprene has a molecular weight from about 5×10² to about 5×10⁶ gmol⁻¹.

4. The method according to claim 1, wherein the proportion by weight of polyisoprene in the total weight of the shaping agent is about 0.01 to about 10 wt. %.

5. The method according to claim 1, wherein the shaping agent has a pH, at 10% solution in water and 20° C., from about 2 to about 12.

6. The method according to claim 1, wherein under conditions of 20° C. and 1013.25 mbar the shaping agent has a viscosity using a Brookfield RTV, spindle 4, and 20 rpm from about 7.5 to about 500 Pas.

7. The method according to claim 1, wherein in step iii. the keratinic fibers undergo a heat treatment using a heat source, wherein the heat source has a temperature from about 50 to about 200° C.

8. The method according to claim 1, wherein the keratinic fibers are straightened in step iii.

9. A shaping agent for keratinic fibers, wherein the shaping agent comprises:
1. keratin-reducing substance(s); and
2. a one polyisoprene.

10. (canceled)

11. The method according to claim 2, wherein the keratin-reducing substance(s) is/are chosen from alkali, and/or ammonium and/or alkanol ammonium salts of sulfurous acid and disulfurous acid.

12. The method according to claim 11, wherein the keratin-reducing substance(s) is/are chosen from sodium sulfite (Na₂SO₃) and/or sodium pyrosulfite (Na₂S₂O₅).

13. The method according to claim 2, wherein the keratin-reducing substance(s) is/are chosen from Bunte salts, thioglycolic acid, thiolactic acid, thiomalic acid, and/or mercaptoethanesulfonic acid and salts and esters thereof.

14. The method according to claim 3, wherein the polyisoprene has a molecular weight from about 10³ to about 4×10⁶ gmol⁻¹.

15. The method according to claim 14, wherein the polyisoprene has a molecular weight from about 10⁵ to about 3×10⁶ gmol⁻¹.

16. The method according to claim 4, wherein the proportion by weight of polyisoprene in the total weight of the shaping agent is about 0.5 to about 6.0 wt. %.

17. The method according to claim 5, wherein the shaping agent has a pH from about 8 to about 10.

18. The method according to claim 6, wherein the shaping agent has a viscosity from about 10 to about 400 Pas.

19. The method according to claim 6, wherein the shaping agent has a viscosity from about 22.5 to about 50 Pas.

20. The method according to claim 7, wherein the heat source has a temperature from about 120 to about 160° C.
